SEQUENCE LISTING (3 sequences)

SEQ ID NO: 1 - DNA, 106 bp, Homo sapiens
gcaaggcactgcaggaggagaaccgcgacctgcgcaaagccagcgtgaccatcgaggatccaaagtgggaattccctcggaagaacttggttcttggaaaaactct

SEQ ID NO: 2 - DNA, 106 bp, Homo sapiens
gcaaggcactgcaggaggagaaccgcgacctgcgcaaagccagcgtgaccatcgaggatccaaagtgggaatttcctcggaagaacttggttcttggaaaaactct

SEQ ID NO: 3 - DNA, 106 bp, Homo sapiens
gcaaggcactgcaggaggagaaccgcgacctgcgcaaagccagcgtgaccatcgaagatccaaagtgggaattccctcggaagaacttggttcttggaaaaactct

(19) United States
(12) Patent Application Publication
(10) Pub. No.: US 2019/0002988 A1
(43) Pub. Date: Jan. 3, 2019

(54) METHOD OF USING A RET FUSION GENE AS A BIOMARKER TO SELECT NON SMALL CELL LUNG CANCER (NSCLC) AND THYROID CANCER PATIENTS FOR A CANCER TREATMENT

(71) Applicant: Boehringer Ingelheim International GmbH, Ingelheim am Rhein (DE)

(72) Inventors: Hidetoshi HAYASHI, Sakai-shi (JP); Kazuhiko NAKAGAWA, Osakasayama-Shi (JP); Kazuto NISHIO, Osakasayama-shi (JP); Kunio OKAMOTO, Takamatsu-shi (JP); Kazuko SAKAI, Habikino-shi (JP); Toshio SHIMIZU, Tokyo (JP); Masayuki TAKEDA, Kawachinagano-shi (JP); Kaoru TANAKA, Sakai-shi (JP)

(21) Appl. No.: 15/781,156

(22) PCT Filed: Dec. 5, 2016
(86) PCT No.: PCT/EP2016/079728

§ 371 Date: Jun. 4, 2018

(30) Foreign Application Priority Data
Dec. 8, 2015 (EP) 15198473.9

(51) Int. Cl. C12Q 1/6886; A61P 35/00; A61K 31/496; A61K 31/337

(52) U.S. Cl. CPC C12Q 1/6886; A61P 35/00; C12Q 2600/156; A61K 31/337; C12Q 2600/106; A61K 31/496

(57) ABSTRACT

The present invention relates to a RET fusion gene such as a CCDC6-RET fusion gene as a biomarker to monitor the activity of the compound 3-Z-[1-(4-(N-((4-methyl-piperazin-1-yl)-methylcarbonyl)-N-methyl-amino)-anilino)-1-phenyl-methylene]-6-methoxycarbonyl-2-indolinone or a pharmaceutically acceptable salt thereof, and especially its monoethanesulphonate salt form, when used alone or optionally in combination with further pharmaceutically active ingredients and/or further treatments. The present invention also relates to specific uses of said specific compound in the treatment of cancers.

The present invention relates to the field of medicine, and especially to a RET fusion gene such as a CCDC6-RET fusion gene as a biomarker of the activity of a specific compound, to the monitoring of a treatment with said compound, and to specific uses of said specific compound in the treatment of cancers.

The present invention relates more specifically to these biomarkers to monitor the activity of the compound 3-Z-[1-(4-(N-((4-methyl-piperazin-1-yl)-methylcarbonyl)-N-methyl-amino)-anilino)-1-phenyl-methylene]-6-methoxycarbonyl-2-indolinone or a pharmaceutically acceptable salt thereof, and especially its monoethanesulphonate salt form, when used alone or optionally in combination with further pharmaceutically active ingredients and/or further treatments, such as for example additional chemotherapy, e.g. with docetaxel.

### BACKGROUND TO THE INVENTION

Recent advances in the knowledge of molecular processes in an organism and techniques to study these processes have resulted in improved methods and systems of typing and treating diseases. Research is being carried out in many fields in order to provide and/or improve methods for the treatment of diseases as well as providing and/or improving methods and systems for monitoring the effects of treatments.

The compound 3-Z-[1-(4-(N-((4-methyl-piperazin-1-yl)-methylcarbonyl)-N-methyl-amino)-anilino)-1-phenyl-methylene]-6-methoxycarbonyl-2-indolinone is an innovative active ingredient having valuable pharmacological properties, especially for the treatment of oncological diseases, immunologic diseases or pathological conditions involving an immunologic component, or fibrotic diseases.

The chemical structure of this compound is depicted below as Formula A.

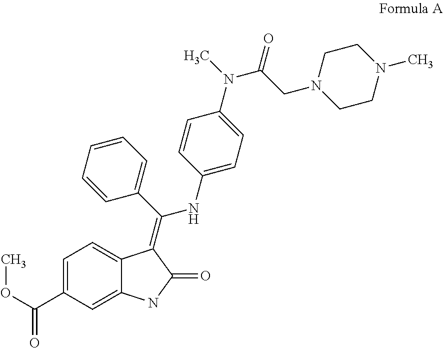 [SMILES: COC(=O)c1ccc2c(c1)NC(=O)/C2=C(\Nc1ccc(N(C)C(=O)CN2CCN(C)CC2)cc1)c1ccccc1]

The base form of this compound is described in WO 01/27081, the monoethanesulphonate salt form is described in WO 2004/013099 and various further salt forms are presented in WO 2007/141283. The use of this molecule for the treatment of immunologic diseases or pathological conditions involving an immunologic component is being described in WO 2004/017948, the use for the treatment of oncological diseases is being described in WO 2004/096224 and the use for the treatment of fibrotic diseases is being described in WO 2006/067165. Uses of this compound are also described in WO 2007/057397.

The monoethanesulphonate salt form of this compound presents properties which makes this salt form especially suitable for development as medicament. The chemical structure of 3-Z-[1-(4-(N-((4-methyl-piperazin-1-yl)-methylcarbonyl)-N-methyl-amino)-anilino)-1-phenyl-methylene]-6-methoxycarbonyl-2-indolinone-monoethanesulphonate is depicted below as Formula A1.

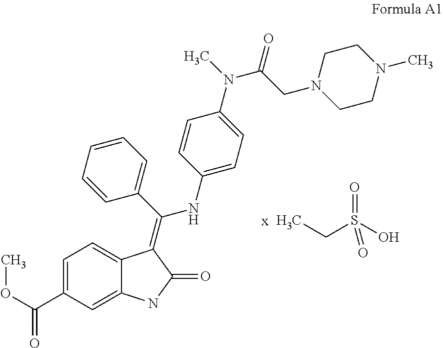

Preclinical studies have shown that this compound is a highly potent, orally bioavailable inhibitor of vascular endothelial growth factor receptors (VEGFRs), platelet-derived growth factor receptors (PDGFRs) and fibroblast growth factor receptors (FGFRs), that suppresses tumor growth through mechanisms inhibiting tumor neovascularization. It has further been shown that this compound inhibits signalling in endothelial- and smooth muscle cells and pericytes, and reduces tumor vessel density.

Furthermore, this compound shows in vivo anti-tumor efficacy in all models tested so far at well tolerated doses. The following Table 1 shows the results of the in vivo anti-tumor efficacy testing in xenograft models and in a syngeneic rat tumor model.

TABLE 1

| Cancer | Model | Efficacy |
|---|---|---|
| Colorectal | HT-29 | T/C 16% @ 100 mg/kg/d |
| | HT-29 large | tumor volume reduction |
| | tumors | |
| Glioblastoma | GS-9L syngeneic | T/C 32% @ 50 mg/kg/d |
| | rat | |
| Head and neck | FaDu | T/C 11% @ 100 mg/kg/d |
| Lung (non-small-cell) | NCI-H460 | T/C 54% @ 25 mg/kg/d |
| | Calu-6 | T/C 24% @ 50 mg/kg/d |
| Ovarian | SKOV3 | T/C 19% @ 50 mg/kg/d |
| Prostate (hormone- | PAC-120 | T/C 34% @ 100 mg/kg/d |
| dependent) | | |
| Renal | Caki-1 | T/C 13% @ 100 mg/kg/d |
| Pancreas (murine | Rip-Tag | interference with tumor |
| transgenic) | | formation |

T/C represents the reduction of tumor size in % of the control

This compound is thus for example suitable for the treatment of diseases in which angiogenesis or the proliferation of cells is involved.

This compound is further suitable for the treatment of fibrotic diseases, as described in WO 2006/067165.

Despite much research aiming at developing methods for diagnosis and screening, there remains a need for efficient methods for the monitoring of treatment, in particular the treatment of cancer. One such method is described in WO 2010/103058.

Moreover, there is little knowledge on, and a need for, biomarkers for monitoring the treatment of a patient with the above-mentioned active ingredient 3-Z-[1-(4-(N-((4-methyl-piperazin-1-yl)-methylcarbonyl)-N-methyl-amino)-anilino)-1-phenyl-methylene]-6-methoxycarbonyl-2-indolinone or a pharmaceutically acceptable salt thereof, and especially its monoethanesulphonate salt form, when used alone or optionally in combination with further pharmaceutically active ingredients and/or further treatments, such as for example radiotherapy or additional chemotherapy.

Additionally, methods which allow the physician and the patient to monitor the current treatment state of the respective cancer (e.g. its response to the treatment, whether or not it has started progressing) and which allow to predict the outcome of the treatment are needed.

The RET fusion gene was identified as a new druggable driver gene present in 1-2% of lung adenocarcinomas (Kohno T, Ichikawa H, Totoki Y et al. KIF5B-RET fusions in lung adenocarcinoma. Nat Med 2012; 18: 375-377), and several clinical trials are under way to address the therapeutic effects of RET tyrosine kinase inhibitors, such as vandetanib and cabozantinib in patients with RET fusion-positive NSCLC (Drilon A, Wang L, Hasanovic A et al. Response to Cabozantinib in patients with RET fusion-positive lung adenocarcinomas. Cancer Discov 2013; 3: 630-635.); however, no selective RET inhibitors have been available in clinical practice.

WO 2014/165710 and Zhang et al., “An evaluation and recommendation of the optimal methodologies to detect RET gene rearrangements in papillary thyroid carcinoma.”, Genes Chromosomes Cancer. 2015 March; 54(3):168-76 both relate to the detection of RET rearrangements.

In view of the art, there remains a need for improved cancer treatments, in particular more efficient cancer therapies which specifically target particular sub-types of cancers and the oncogenic pathways promoting these sub-types of cancers.

Thus, the present invention provides methods to monitor a cancer treatment and to predict the outcome of a cancer treatment, in particular the treatment of non-small cell lung cancer or thyroid cancer with the compound 3-Z-[1-(4-(N-((4-methyl-piperazin-1-yl)-methylcarbonyl)-N-methyl-amino)-anilino)-1-phenyl-methylene]-6-methoxycarbonyl-2-indolinone or a pharmaceutically acceptable salt thereof.

Moreover, the inventors have surprisingly found that patients positive for the RET fusion gene can more advantageously be treated by said compound or pharmaceutically acceptable salt thereof. Thus, the invention also provides specific medical uses that allow to advantageously treat a specific form of cancer that contains the RET fusion gene.

### DESCRIPTION OF THE INVENTION

The inventors have unexpectedly found that a RET fusion gene, more particularly the CCDC6-RET fusion gene, is present in NSCLC patients that are very susceptible to therapy with a compound according to the invention, 3-Z-[1-(4-(N-((4-methyl-piperazin-1-yl)-methylcarbonyl)-N-methyl-amino)-anilino)-1-phenyl-methylene]-6-methoxycarbonyl-2-indolinone. Notably, in a patient who was tested for multiple cancerous mutations, it was found that said RET fusion gene was present, and that no other actionable mutations were present.

Accordingly, in a non-limiting aspect of the invention, a RET fusion gene such as the CCDC6-RET fusion gene can be used as a cancer biomarker. More particularly, by determining whether a RET fusion gene is present in cancers such as thyroid cancer or non-small cell lung cancer according to the present invention, the cancer treatment can be monitored, and the treatment outcome of the cancer treatment can be predicted, as reflected in the following preferred embodiments.

Furthermore, given that the RET protein is inhibited by a compound according to the invention, 3-Z-[1-(4-(N-((4-methyl-piperazin-1-yl)-methylcarbonyl)-N-methyl-amino)-anilino)-1-phenyl-methylene]-6-methoxycarbonyl-2-indolinone, patients containing a RET fusion gene will particularly benefit from the treatment, because in addition to the inhibition of VEGF signalling, the treatment with the compound according to the invention will also inhibit the RET fusion protein. Thus, unexpectedly, in patients containing a RET fusion gene, two oncogenic pathways will advantageously be inhibited by the same compound.

Thus, the invention also encompasses compounds for the medical uses which are reflected in the preferred embodiments defined below.

### Preferred Embodiments

1. A method for selecting a thyroid cancer or non-small cell lung cancer patient for a treatment with the compound 3-Z-[1-(4-(N-((4-methyl-piperazin-1-yl)-methylcarbonyl)-N-methyl-amino)-anilino)-1-phenyl-methylene]-6-methoxycarbonyl-2-indolinone or a pharmaceutically acceptable salt thereof, said method comprising the steps of
a) determining whether a RET fusion gene and/or a RET fusion gene product is present in a sample from said patient; and
b) selecting said patient for said treatment if said RET fusion gene and/or a RET fusion gene product is determined to be present in said sample.
   2. A method for predicting the clinical outcome of a thyroid cancer or non-small cell lung cancer treatment of a patient with the compound 3-Z-[1-(4-(N-((4-methyl-piperazin-1-yl)-methylcarbonyl)-N-methyl-amino)-anilino)-1-phenyl-methylene]-6-methoxycarbonyl-2-indolinone or a pharmaceutically acceptable salt thereof, said method comprising a step of determining whether a RET fusion gene and/or a RET fusion gene product is present in a sample from said patient.
   3. The method of item 1 or 2, wherein the compound is the monoethanesulphonate salt form of 3-Z-[1-(4-(N-((4-methyl-piperazin-1-yl)-methylcarbonyl)-N-methyl-amino)-anilino)-1-phenyl-methylene]-6-methoxycarbonyl-2-indolinone.
   4. The method of any one of items 1 to 3, further comprising the step of predicting that, if said RET fusion gene and/or RET fusion gene product is determined to be present in said sample, the patient will have a clinical outcome which is:
a prolonged response to said treatment;
a prolonged stabilisation under said treatment;
a prolonged time to cancer progression under said treatment;
a prolonged progression free survival; and/or
a prolonged overall survival
   as compared to a patient where said RET fusion gene and/or RET fusion gene product is determined to be absent.
   5. The method of item 4, wherein said clinical outcome is a prolonged time to cancer progression under said treatment.
   6. The method of any one of items 1 to 5, wherein the sample is a tumor biopsy of said cancer obtained from said patient.
   7. The method of any one of items 1 to 5, wherein the sample is blood obtained from said patient or a blood fraction obtained from said patient.
   8. The method of item 7, wherein the sample is blood obtained from said patient.
   9. The method of any one of items 1 to 8, wherein said RET fusion gene is CCDC6-RET, and wherein said RET fusion gene product is the product of the CCDC6-RET fusion gene.
   10. The method of any one of items 1 to 9, wherein said cancer is non-small cell lung cancer.
   11. The method of any one of items 1 to 9, wherein said cancer is thyroid cancer.
   12. The method of any one of items 1 to 11, wherein said treatment is a monotherapy with said 3-Z-[1-(4-(N-((4-methyl-piperazin-1-yl)-methylcarbonyl)-N-methyl-amino)-anilino)-1-phenyl-methylene]-6-methoxycarbonyl-2-indolinone or said pharmaceutically acceptable salt thereof.
   13. The method of any one of items 1 to 11, wherein said treatment is a combination therapy with
i) said 3-Z-[1-(4-(N-((4-methyl-piperazin-1-yl)-methylcarbonyl)-N-methyl-amino)-anilino)-1-phenyl-methylene]-6-methoxycarbonyl-2-indolinone or said pharmaceutically acceptable salt thereof; and
ii) an additional anticancer therapy, preferably therapy with docetaxel.
   14. The method of any one of items 1 to 13, wherein said RET fusion gene and/or RET fusion gene product is the fusion gene CCDC6-RET.
   15. The method of any one of items 1 to 14, wherein said step of determining comprises a step of
forming a detection complex of said RET fusion gene with a reagent for detecting said RET fusion gene, said reagent preferably being a nucleic acid capable of specific binding to said RET fusion gene;
   and/or wherein said step of determining comprises a step of
forming a detection complex of said RET fusion gene product with a reagent for detecting said RET fusion gene product, wherein said RET fusion gene product is a fusion protein, and wherein said reagent preferably is an antibody capable of specific binding to said fusion protein;
   and/or wherein said step of determining comprises a step of
forming a detection complex of said RET fusion gene product with a reagent for detecting said RET fusion gene product, wherein said RET fusion gene product is an mRNA of the RET fusion gene, and wherein said reagent preferably is at least one nucleic acid capable of specific binding to said mRNA of the RET fusion gene.
   16. A method of any one of the preceding items, wherein said method is an in vitro method.
   17. A method to determine whether a thyroid cancer or non-small cell lung cancer treatment of a patient with the compound 3-Z-[1-(4-(N-((4-methyl-piperazin-1-yl)-methylcarbonyl)-N-methyl-amino)-anilino)-1-phenyl-methylene]-6-methoxycarbonyl-2-indolinone or a pharmaceutically acceptable salt thereof is or will be effective, comprising the following elements: a patient or doctor requesting such a determination; acquisition of a sample from said patient; analysis of the sample using a method according to any one of the preceding items; and communication of the tests results back to the patient or doctor.
   18. A diagnostic kit comprising at least one means for performing a method according to any one of items 1 to 15.
   19. The diagnostic kit of item 18, wherein the kit comprises a material selected from:
at least one antibody capable of specific binding to said RET fusion gene product or an antigen-binding fragment thereof; and/or
at least one nucleic acid capable of specific binding to said RET fusion gene or capable of specific binding to a RET fusion gene product, said RET fusion gene product being an mRNA of the RET fusion gene;
   the kit optionally further comprising instructions for use including instructions for performing the method according to any one of items 1 to 14.
   20. The diagnostic kit of item 19, wherein the kit comprises at least one nucleic acid capable of specific binding to said RET fusion gene or capable of specific binding to a RET fusion gene product, said RET fusion gene product being an mRNA of the RET fusion gene, and
   wherein said at least one nucleic acid comprises a nucleic acid sequence that is complementary to a sequence represented by SEQ ID No. 1, SEQ ID No. 2 or SEQ ID No. 3 or complementary to a reverse complement thereof.
   21. Use of a RET fusion gene and/or a RET fusion gene product as a biomarker for selecting a thyroid cancer or non-small cell lung cancer patient for a treatment with the compound 3-Z-[1-(4-(N-((4-methyl-piperazin-1-yl)-methylcarbonyl)-N-methyl-amino)-anilino)-1-phenyl-methylene]-6-methoxycarbonyl-2-indolinone or a pharmaceutically acceptable salt thereof.
   22. The use of item 21, wherein the use is an in vitro use.
   23. The use of item 21 or 22, wherein said RET fusion gene and/or RET fusion gene product is as defined in any one of items 9 or 14.
   24. The compound 3-Z-[1-(4-(N-((4-methyl-piperazin-1-yl)-methylcarbonyl)-N-methyl-amino)-anilino)-1-phenyl-methylene]-6-methoxycarbonyl-2-indolinone or a pharmaceutically acceptable salt thereof, for use in a method for the treatment of cancer in a patient, wherein said patient contains a RET fusion gene.
   25. The compound 3-Z-[1-(4-(N-((4-methyl-piperazin-1-yl)-methylcarbonyl)-N-methyl-amino)-anilino)-1-phenyl-methylene]-6-methoxycarbonyl-2-indolinone or a pharmaceutically acceptable salt thereof, for use in a method for the treatment of cancer in a patient, wherein the patient is a patient who has been selected according to the method of any one of items 1 and 3 to 16.
   26. The compound or pharmaceutically acceptable salt thereof of item 24 or 25 for the use of item 24 or 25, wherein the cancer is non-small cell lung cancer or thyroid cancer.
   27. The compound or pharmaceutically acceptable salt thereof of any one of items 24 to 26 for the use of any one of items 24 to 26, wherein the method for treatment is a method for the treatment of human patients with locally advanced, metastatic or locally recurrent non-small cell lung cancer (NSCLC) of adenocarcinoma tumour histology after first-line chemotherapy, and wherein the patient is preferably an adult patient.
   28. The compound or pharmaceutically acceptable salt thereof of any one of items 24 to 27 for the use of any one of items 24 to 27, wherein said RET fusion gene is present in blood obtained from said patient, in a blood fraction obtained from said patient and/or in a tumor biopsy obtained from said cancer of said patient.
   29. The compound or pharmaceutically acceptable salt thereof of any one of items 24 to 28 for the use of any one of items 24 to 28, wherein the RET fusion gene is the fusion gene CCDC6-RET.
   30. The compound or pharmaceutically acceptable salt thereof of any one of items 24 to 29 for the use of any one of items 24 to 29, wherein said cancer is non-small cell lung cancer.
   31. The compound or pharmaceutically acceptable salt thereof of any one of items 24 to 30 for the use of any one of items 24 to 30, wherein the use is a use for inhibiting the protein product of said RET fusion gene.
   32. The compound or pharmaceutically acceptable salt thereof of any one of items 24 to 31 for the use of any one of items 24 to 31, wherein said cancer is non-small cell lung cancer, preferably advanced non-small cell lung cancer, and wherein the compound or pharmaceutically acceptable salt thereof is to be administered over an administration period of at least 18 months, preferably at least 24 months, more preferably at least 30 months.
   33. The compound or pharmaceutically acceptable salt thereof of any one of items 24 to 32 for the use of any one of items 24 to 32, wherein the compound is the monoethanesulphonate salt form of 3-Z-[1-(4-(N-((4-methyl-piperazin-1-yl)-methylcarbonyl)-N-methyl-amino)-anilino)-1-phenyl-methylene]-6-methoxycarbonyl-2-indo linone.
   34. The compound or pharmaceutically acceptable salt thereof of any one of items 24 to 33 for the use of any one of items 22 to 33, wherein the compound is used in combination with an additional anticancer agent, preferably docetaxel.

### BRIEF DESCRIPTION OF THE DRAWINGS

FIG. 1: Chest CT scans of the patient show the patient's disease burden before treatment, after a response to nintedanib, and at the time of nintedanib resistance.

FIG. 2: Junction reads for the CCDC6-RET fusion transcripts. Nucleotides are indicated by different colors. The junction reads span the junctions within the gene fusion. Read sequences for CCDC6-RET in tumor specimens were visualized with the Integrative Genomics Viewer.

FIG. 3: A break-apart fluorescence in situ hybridization (FISH) assay with a 5′ RET probe (green) and a 3′ RET probe (red) shows the RET rearrangement, as indicated by the presence of single isolated red 3′ RET probes (arrowheads). The normal RET locus is shown as unsplit red and green pairs of probes (arrows). The nuclei are stained with 4′,6-diamidino-2-phenylindole.

FIG. 4:

For DNA sequencing, 10 ng of DNA were subjected to multiplex PCR amplification with the use of an Ion AmpliSeq Library Kit 2.0 and Ion AmpliSeq Colon and Lung Cancer Panel (Life Technologies), that targets 504 mutational hotspot regions of 22 cancer-associated genes.

FIG. 5:

A) and B): For RNA sequencing, 10 ng of RNA were subjected to reverse transcription with the use of a SuperScript VILO cDNA Synthesis Kit (Life Technologies) followed by library generation with the use of an Ion AmpliSeq Library Kit 2.0 and Ion AmpliSeq RNA Fusion Lung Cancer Research Panel (Life Technologies), the latter of which detects transcripts derived from ALK, RET, ROS1, and NTRK1 fusion genes.

### DETAILED DESCRIPTION OF THE INVENTION

Within the meaning of the present invention, a biomarker is used as an indicator of a biologic state. It is a characteristic that is objectively measured and evaluated as an indicator of normal biologic processes, pathogenic processes, or pharmacologic responses to a therapeutic intervention. This is in line with the definition given by an NIH study group in 1998.

More specifically, a biomarker indicates a change that correlates with the risk or progression of a disease, or with the susceptibility of the disease to a given treatment. Once a proposed biomarker has been validated, it can be used to diagnose disease risk, presence of disease in a human patient, or to tailor treatments for the disease in a human patient (choices of drug treatment or administration regimes). In evaluating potential drug therapies, a biomarker may be used as a surrogate for a natural endpoint such as survival or irreversible morbidity. If a treatment alters the biomarker, which has a direct connection to improved health, the biomarker serves as a surrogate endpoint for evaluating clinical benefit.

The method of the invention can be, for example, an in vitro method wherein after a sample has been taken from the patient, the presence of biomarker(s) (i.e. the RET fusion gene and/or gene product) in the sample is determined. The sample can be any sample that contains the RET fusion gene and/or gene product. That means that the sample may, for instance, be a tumor tissue sample from a tumor formed by the cancer, e.g. a tumor biopsy from a tumor formed by the cancer. Alternatively, the sample may be any other sample that contains the RET fusion gene and/or gene product, e.g. blood (including, without limitation, whole fresh blood, and frozen whole blood) or a blood fraction (including, without limitation, serum, fresh plasma, or frozen plasma).

As shown in the following and in accordance with the present invention, RET fusion genes are useful as biomarkers to monitor the cancer treatment of a patient with the active ingredient 3-Z-[1-(4-(N-((4-methyl-piperazin-1-yl)-methylcarbonyl)-N-methyl-amino)-anilino)-1-phenyl-methylene]-6-methoxycarbonyl-2-indolinone or a pharmaceutically acceptable salt thereof, preferably the monoethanesulphonate salt form, to select a patient based on said biomarker, and to predict the clinical outcome of such cancer treatment.

It will be understood that in accordance with the methods in accordance with the invention, terms such as “predicting” or “predicting that [ . . . ] the patient will have a clinical outcome” include predictions with a likelihood of higher than 50%, preferably higher than 60%, more preferably higher than 70%, still more preferably higher than 80%, still more preferably higher than 90%, still more preferably higher than 95%, still more preferably higher than 98%, still more preferably higher than 99%, still more preferably higher than 99.5%, still more preferably higher than 99.8% and still more preferably higher than 99.8%.

Statistical Methods for predicting the clinical outcome based on the presence of particular genotypes are known in the art and can readily be adapted to the methods according to the invention.

As used herein, the term “RET” in connection with fusion genes and their products means any RET fusion gene or gene product known in the art. Most preferably, the RET fusion gene is the fusion gene CCDC6-RET, and its gene product is a gene product of the fusion gene CCDC6-RET, as, for instance, defined in the preferred embodiments.

### Samples Used in the Methods:

In one embodiment of all methods of the invention, several samples from said patient are obtained at one time point after initiation of treatment, or at different time points after initiation of treatment. This enables selecting a patient during the course of treatment. This is for instance useful for establishing appropriate treatment schedules, dosage and type on a patient per patient basis. Furthermore it can be determined whether continuation of treatment at or beyond a given time point is appropriate.

In a preferred embodiment of the present invention in accordance with the above methods, said sample is obtained within a month of initiation of said treatment.

In another preferred embodiment of the present invention in accordance with the above methods, said sample is obtained within a week of initiation of said treatment.

In another preferred embodiment of the present invention in accordance with the above methods, said sample is obtained within two days of initiation of said treatment.

In the methods for predicting the clinical outcome of the invention, said sample can also be obtained at one or more time points before initiation of treatment.

This allows to predict the clinical outcome of the patients based on the presence or absence of a RET fusion gene even before the treatment has started, and to identify, before the start of the treatment, a specific population of patients who are very susceptible to the treatment. Based on the prediction, the treatment may be started either in all patients, or in a group of patients where the RET fusion gene or its gene product is determined to be present.

Thus, in a preferred embodiment of the present invention in accordance with the above methods for predicting the clinical outcome, said sample is obtained within a month before initiation of said treatment.

In another preferred embodiment of the present invention in accordance with the above methods for predicting the clinical outcome, said sample is obtained within a week before initiation of said treatment.

In another preferred embodiment of the present invention in accordance with the above methods for predicting the clinical outcome, said sample is obtained within two days before initiation of said treatment.

As used herein, terms such as “within a month before initiation of said treatment”, “within a week before initiation of said treatment”, and “within two days before initiation of said treatment” are meant to include the day of the treatment, provided that said sample is obtained before initiation of said treatment.

Determining the Presence of a RET Fusion Gene and/or a RET Fusion Gene Product

Methods for determining the presence of fusion genes and their fusion gene products in general are known in the art; and all of these methods can be used for determining a RET fusion gene and/or a RET fusion gene product in accordance with the invention.

For instance, preferred methods that can be used in accordance with the methods of the invention are as disclosed in Zhang et al., “An evaluation and recommendation of the optimal methodologies to detect RET gene rearrangements in papillary thyroid carcinoma.”, Genes Chromosomes Cancer. 2015 March; 54(3):168-76, which is hereby incorporated by reference.

Further preferred methods that can be used in accordance with the methods of the invention are disclosed in WO 2014/165710.

In accordance with the invention, the presence of a RET fusion gene may be determined directly (i.e. by a using a method that determines the gene itself), and/or indirectly by determining the presence of a RET fusion gene product such as an mRNA of a RET fusion gene or a protein product of a RET fusion gene.

Methods to determine a RET fusion gene itself include any suitable DNA detection methods such as Southern Blotting techniques, fluorescence in situ hybridization (FISH) (as, for instance, demonstrated in Example 1), DNA arrays and DNA sequencing-based methods.

Methods to determine a RET fusion gene product include any suitable mRNA detection methods such as Northern Blotting techniques, RNA arrays and RNA sequencing-based methods.

Methods to determine a RET fusion gene product also include methods to determine the fusion protein, such as antibody-based methods.

In accordance with the invention, any nucleic acid detection-based techniques may be preceded by suitable amplification steps. For instance, RNA sequencing-based methods to detect a RET fusion gene product may be preceded by reverse transcription, library generation and multiplex PCR amplification. Such amplification is, for instance, exemplified in Example 1.

Cancers in Accordance with the Invention

The present invention as defined herein is of interest for any cancers, for which RET fusion genes can be identified in particular groups of patients. Such cancers according to the invention are preferably non-small cell lung cancer and thyroid cancer, more preferably non-small cell lung cancer.

### Medical Uses of the Invention:

In the treatments in accordance with the invention, the above-mentioned compounds according to the invention (i.e. 3-Z-[1-(4-(N-((4-methyl-piperazin-1-yl)-methylcarbonyl)-N-methyl-amino)-anilino)-1-phenyl-methylene]-6-methoxycarbonyl-2-indolinone or a pharmaceutically acceptable salt thereof) may be administered as a monotherapy or as a combination therapy.

As used herein, the term “monotherapy” refers to a treatment with a compound of the invention without an additional anticancer therapy such as radiotherapy or chemotherapy.

A “combination therapy” of the present invention as defined herein may be achieved by way of the simultaneous, sequential or separate administration of the individual components of said treatment. A combination treatment as defined herein may be applied as a sole therapy or may involve surgery or radiotherapy or an additional chemotherapeutic or targeted agent in addition to a combination treatment of the invention. Surgery may comprise the step of partial or complete tumour resection, prior to, during or after the administration of the combination treatment as described herein.

Combination treatments of the present invention are expected to be particularly useful in the treatment cancer such as non-small cell lung cancer (NSCLC), and thyroid cancer.

More particularly such combination treatments of the invention are expected to inhibit any form of cancer associated with VEGF and RET including non-small cell lung cancer (NSCLC), and thyroid cancer. More especially combination treatments of the present invention are expected to slow advantageously the growth of tumors in non-small cell lung cancer (NSCLC).

The following studies are intended to illustrate the present invention and do not limit the invention.

### Phase I Clinical Study

### Study Outline:

The present study is a case study of long-term efficacy of docetaxel plus nintedanib (BIBF 1120; 3-Z-[1-(4-(N-((4-methyl-piperazin-1-yl)-methylcarbonyl)-N-methyl-amino)-anilino)-1-phenyl-methylene]-6-methoxycarbonyl-2-indolinone) over 33 months in a never smoking woman with EGFR and ALK wild-type non-small cell lung cancer NSCLC. Multiplex genetic testing has revealed CCDC6-RET fusion gene in lung biopsy specimens, and no other actionable mutations were identified. Thus, in a non-limiting aspect of the invention, the inventors have found that RET rearrangement correlates with the sensitivity to nintedanib.

### Case Presentation:

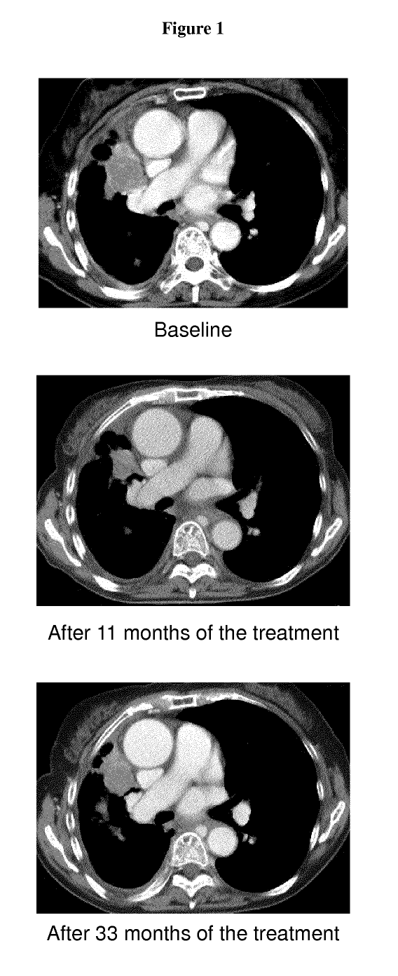

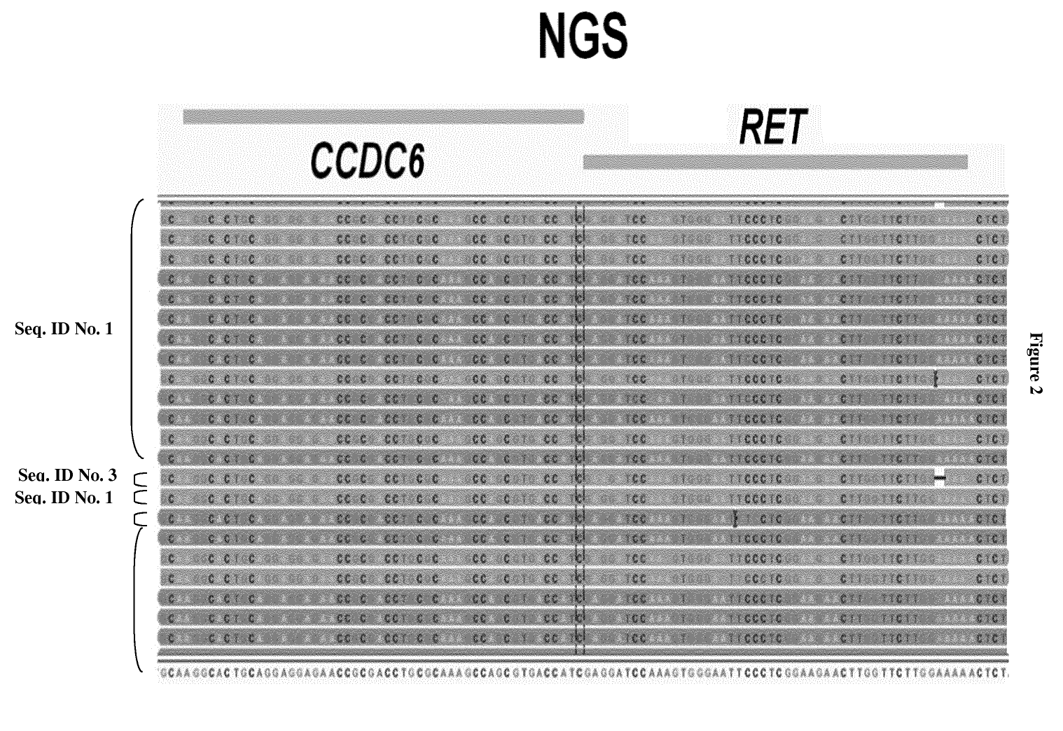

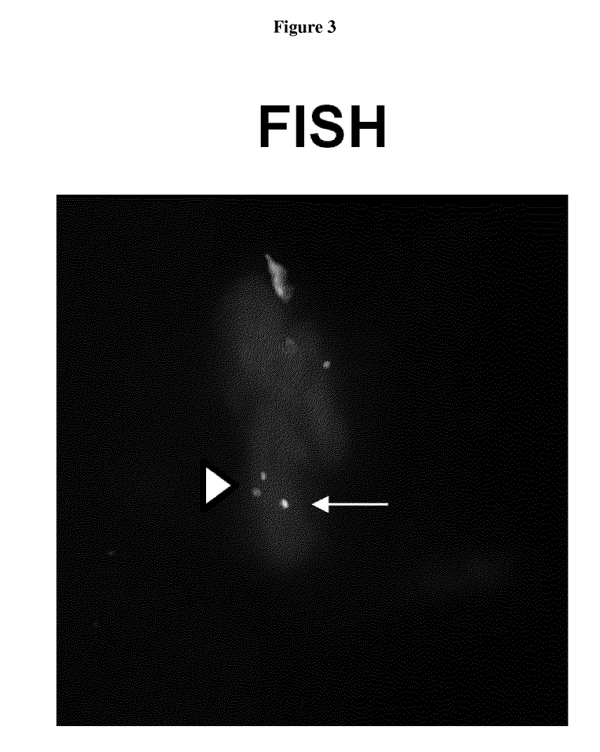

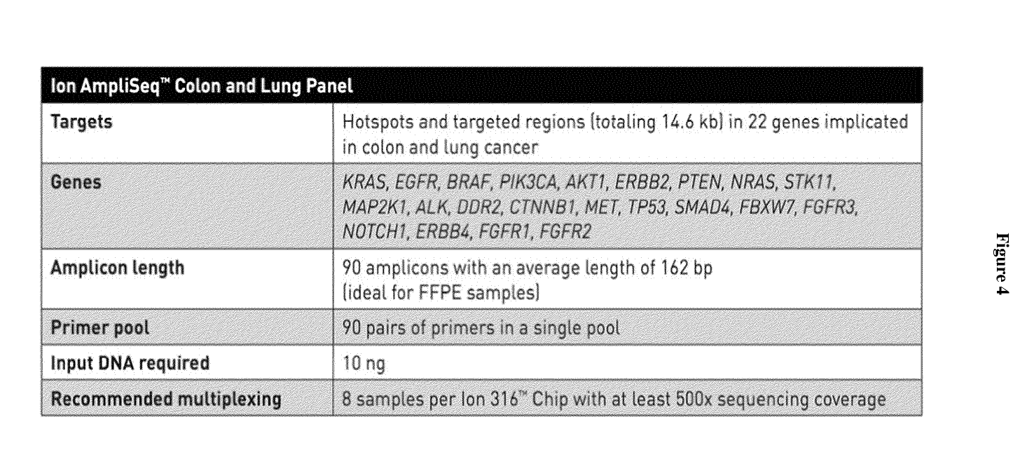

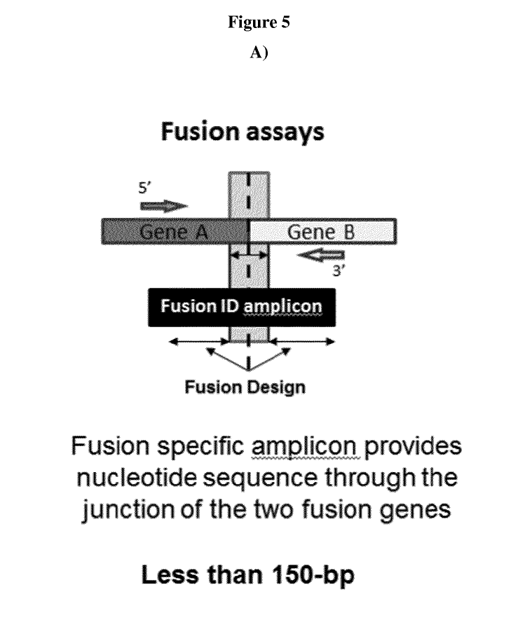

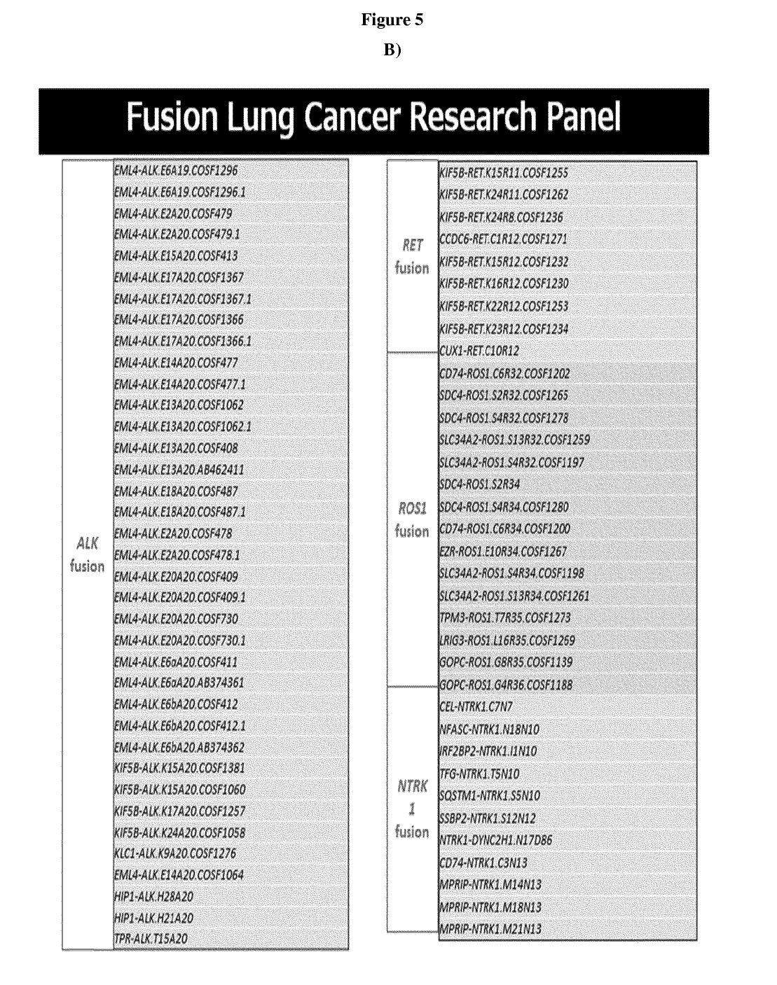

The present case was a 60 year-old never smoking woman with EGFR and ALK wild-type advanced NSCLC. After failure of first-line platinum-based chemotherapy, the patient was enrolled in a phase I dose-escalation study of nintedanib combined with docetaxel for Japanese patients with advanced NSCLC on June, 2010, with oral administration of nintedanib 150 mg twice daily plus docetaxel 75 mg/m² every 3 weeks (Okamoto I, Kaneda H, Satoh T et al. Phase I safety, pharmacokinetic, and biomarker study of BIBF 1120, an oral triple tyrosine kinase inhibitor in patients with advanced solid tumors. Mol Cancer Ther 2010; 9: 2825-2833.). Although the patient had a radiographic response to docetacel plus nintedanib, docetaxel was discontinued after 22 cycles due to lower extremity edema and fatigue, and nintedanib monotherapy was continued afterwards. A total of 48 treatment cycles (26 cycles of therapy with nintedanib alone) had been administered over 33 months until disease progression on March, 2013 (FIG. 1). To identify mutations which are highly susceptible to therapy with nintedanib, next-generation sequencing panels were applied that cover both mutational hotspots in 22 genes related to lung and colon tumorigenesis using DNA (FIG. 4) as well as 72 major variants of ALK, RET, ROS1, and NTRK1 fusion transcripts using RNA extracted from non-small lung tissues obtained by transbronchial biopsy (FIG. 5). Multiplex genetic testing has identified CCDC6-RET fusion gene in tissue specimens for lung cancer diagnosis (FIG. 2), and no other actionable mutations were identified. This chromosomal inversion was verified using fluorescence in situ hybridization, which revealed a split in the signals for the probes that flank the RET translocation sites in tumors positive for the CCDC6-RET fusion (FIG. 3).

The proof of principle for VEGF inhibition in NSCLC has been demonstrated in clinical trials of bevacizumab. Nintedanib is have been developed to have more potent selectivity for VEGFR kinase, and preclinical studies with nintedanib have shown sustained blockade of VEGFR2 in vitro, and delay or arrest of tumor growth in xenograft models of human solid tumors (Hilberg F, Roth G J, Krssak M et al. BIBF 1120: triple angiokinase inhibitor with sustained receptor blockade and good antitumor efficacy. Cancer Res 2008; 68: 4774-4782.). Given the biological relevance of anti-angiogenic activity in lung cancer, the phase III trial assessed nintedanib plus docetaxel versus docetaxel alone in patients with advanced NSCLC with any histology, and demonstrated that treatment with a combination of nintedanib and docetaxel significantly and clinically meaningful improved overall survival compared with docetaxel alone in the predefined patients with adenocarcinoma tumor histology (Reck M, Kaiser R, Mellemgaard A et al. Docetaxel plus nintedanib versus docetaxel plus placebo in patients with previously treated non-small-cell lung cancer (LUME-Lung 1): a phase 3, double-blind, randomised controlled trial. Lancet Oncol 2014; 15: 143-155.); however, tumor samples have not been collected to identify the biological rationale underpinning the response to nintedanib in combination with docetaxel in NSCLC.

In this study, multiplex testing was conducted to identify mutations potentially predictive of response in patient who show the long-term efficacy of nintedanib, and it identified CCDC6-RET fusion without other activating mutations. Nintedanib reportedly inhibits receptor kinases of RET in addition to previously noted anti-angiokinases (VEGFR1-3, FGFR1-3, PDGFR α and β) in vitro (Hilberg F, Roth G J, Krssak M et al. BIBF 1120: triple angiokinase inhibitor with sustained receptor blockade and good antitumor efficacy. Cancer Res 2008; 68: 4774-4782.). Thus, according to the invention, the presence of RET fusions such as CCDC6-RET fusion (e.g. when identified by multiplex testing) in cancers correlates with the sensitivity to nintedanib. Therefore, RET rearrangement can advantageously be used to monitor the treatment of cancers with nintedanib or pharmaceutically active salts thereof, in particular cancers containing RET rearrangements such as thyroid cancer and non-small cell lung cancer (NSCLC), and to select patients for treatments with the compounds of the invention. Moreover, according to the invention, these cancers can be treated more effectively with nintedanib or pharmaceutically active salts thereof if they contain a RET rearrangement, for instance a RET fusion gene such as the CCDC6-RET fusion gene. Unexpectedly, such treatments can be carried out for an extended period of time (e.g. for at least 18 months, for at least 24 months or even for at least 30 months for NSCLC), since cancer progression is delayed under such treatments.

The following detection methods were used for the study:

### Fusion Gene Detection Using NGS

RNA was purified with the use of an Allprep DNA/RNA FFPE Kit (Qiagen, Valencia, Calif.) and were then subjected to NGS panels for fusion transcript detection. Ten ng of RNA were subjected to reverse transcription with the use of a SuperScript VILO cDNA Synthesis Kit (Life Technologies) followed by library generation with the use of an Ion AmpliSeq Library Kit 2.0 and Ion AmpliSeq RNA Fusion Lung Cancer Research Panel (Life Technologies), the latter of which detects transcripts derived from 37 ALK, 9 RET, 15 ROSJ, and 11 NTRK1 fusion genes as well as reference sequences for 5′ and 3′ expression imbalance and five housekeeping genes (HMBS, TBP, ITGB7, MYC, LMNA). After multiplex PCR, Ion Xpress Barcode Adapters (Life Technologies) were ligated to the PCR products, which were then purified with the use of Agencourt AMPure XP beads (Beckman Coulter, Brea, Calif.). The purified libraries were pooled and then sequenced with the use of an Ion Torrent PGM instrument, Ion PGM 200 Sequencing Kit v2, and Ion 318 v2 Chip Kit (all from Life Technologies). Unaligned bam files were transferred to Ion Reporter Software 4.2 (Life Technologies) and analyzed for the number of counts per amplicon in each sample. Fusion genes were judged positive when both of following criteria were met: for ALK, a fusion read count of >100 and 3′/5′ imbalance value of >0.025; for RET, a fusion read count of >100 and 3′/5′ imbalance value of >0.045; and for ROS1, a fusion read count of >100 and 3′/5′ imbalance value of >0.5.

### FISH Analysis

FFPE tissue sectioned at a thickness of 4 μm and placed on a glass slide was subjected to FISH analysis with break-apart probes for RET (Split Dual Color FISH Probes SP019; GSP Laboratory, Kawasaki, Japan). FISH positivity was defined as the presence of >15% split signals in tumor cells.

### Further Embodiments

Further pharmaceutically acceptable salts of the compound 3-Z-[1-(4-(N-((4-methyl-piperazin-1-yl)-methylcarbonyl)-N-methyl-amino)-anilino)-1-phenyl-methylene]-6-methoxycarbonyl-2-indolinone than those already described hereinbefore may, for example, include acid addition salts. Such acid addition salts include, for example, salts with inorganic or organic acids affording pharmaceutically acceptable anions such as with hydrogen halides or with sulphuric or phosphoric acid, or with trifluoroacetic, citric or maleic acid. In addition, pharmaceutically acceptable salts may be formed with an inorganic or organic base which affords a pharmaceutically acceptable cation. Such salts with inorganic or organic bases include for example an alkali metal salt, such as a sodium or potassium salt and an alkaline earth metal salt such as a calcium or magnesium salt.

In accordance with the present invention, the compounds may be formulated using one or more pharmaceutically acceptable excipients or carriers, as suitable. Suitable formulations which may be used within the scope of the present invention have already been described in the literature and in patent applications related to these compounds WO 2009/147212 and WO 2009/147220. These formulations are incorporated herein by reference.

In one embodiment in accordance with the present invention, the formulation for the compound of formula A1 is a lipid suspension of the active substance comprising preferably a lipid carrier, a thickener and a glidant/solubilizing agent, most preferably in which the lipid carrier is selected from corn oil glycerides, diethylenglycolmonoethylether, ethanol, glycerol, glycofurol, macrogolglycerolcaprylocaprate, macrogolglycerollinoleate, medium chain partial glycerides, medium chain triglycerides, polyethylene glycol 300, polyethylene glycol 400, polyethylene glycol 600, polyoxyl castor oil, polyoxyl hydrogenated castor oil, propylene glycol monocaprylate, propylene glycol monolaurate, refined soybean oil, triacetin, triethyl citrate, or mixtures thereof, the thickener is selected from oleogel forming excipients, such as Colloidal Silica or Bentonit, or lipophilic or amphiphilic excipients of high viscosity, such as polyoxyl hydrogenated castor oil, hydrogenated vegetable oil macrogolglycerol-hydroxystearates, macrogolglycerol-ricinoleate or hard fats, and the glidant/solubilizing agent is selected from lecithin, optionally further comprising one or more macrogolglycerols, preferably selected from macrogolglycerol-hydroxystearate or macrogolglycerol-ricinoleate. The lipid suspension formulation may be prepared by conventional methods of producing formulations known from the literature, i.e. by mixing the ingredients at a pre-determined temperature in a pre-determined order in order to obtain a homogenized suspension.

The above formulation may be preferably incorporated in a pharmaceutical capsule, preferably a soft gelatin capsule, characterised in that the capsule shell comprises e.g. glycerol as plasticizing agent, or a hard gelatin or hydroxypropylmethylcellulose (HPMC) capsule, optionally with a sealing or banding. The capsule pharmaceutical dosage form may be prepared by conventional methods of producing capsules known from the literature. The soft gelatin capsule may be prepared by conventional methods of producing soft gelatin capsules known from the literature, such as for example the “rotary die procedure”, described for example in Swarbrick, Boylann, Encyclopedia of pharmaceutical technology, Marcel Dekker, 1990, Vol. 2, pp 269 ff or in Lachmann et al., “The Theory and Practice of Industrial Pharmacy”, 2nd Edition, pages 404-419, 1976, or other procedures, such as those described for example in Jimerson R. F. et al., “Soft gelatin capsule update”, Drug Dev. Ind. Pharm., Vol. 12, No. 8-9, pp. 1133-44, 1986.

The above defined formulation or the above defined capsule may be used in a dosage range of from 0.1 mg to 20 mg of active substance/kg body weight, preferably 0.5 mg to 4 mg active substance/kg body weight.

The above defined capsules may be packaged in a suitable glass container or flexible plastic container, or in an aluminium pouch or double poly bag.

The dosages and schedules may vary according to the particular disease state and the overall condition of the patient. Dosages and schedules may also vary if, in addition to a treatment with compound A of the present invention or a pharmaceutically acceptable salt thereof, one or more additional chemotherapeutic agents is/are used. Scheduling can be determined by the practitioner who is treating any particular patient.

Radiotherapy may be administered according to the known practices in clinical radiotherapy. The dosages of ionising radiation will be those known for use in clinical radiotherapy. The radiation therapy used will include for example the use of γ-rays, X-rays, and/or the directed delivery of radiation from radioisotopes. Other forms of DNA damaging factors are also included in the present invention such as microwaves and UV-irradiation. For example X-rays may be dosed in daily doses of 1.8-2.0 Gy, 5 days a week for 5-6 weeks. Normally a total fractionated dose will lie in the range 45-60 Gy. Single larger doses, for example 5-10 Gy may be administered as part of a course of radiotherapy. Single doses may be administered intraoperatively. Hyperfractionated radiotherapy may be used whereby small doses of X-rays are administered regularly over a period of time, for example 0.1 Gy per hour over a number of days. Dosage ranges for radioisotopes vary widely, and depend on the half-life of the isotope, the strength and type of radiation emitted, and on the uptake by cells.

The size of the dose of each therapy which is required for the therapeutic or prophylactic treatment of a particular disease state will necessarily be varied depending on the host treated, the route of administration and the severity of the illness being treated. Accordingly the optimum dosage may be determined by the practitioner who is treating any particular patient. For example, it may be necessary or desirable to reduce the above-mentioned doses of the components of the combination treatments in order to reduce toxicity.

### Use of Nintedanib for NSCLC

The recommended dose of nintedanib is 200 mg (amount calculated on free base, administered as esilate) twice daily administered approximately 12 hours apart, on days 2 to 21 of a standard 21 day docetaxel treatment cycle. Nintedanib must not be taken on the same day of docetaxel chemotherapy administration (=day 1). If a dose of nintedanib is missed, administration should resume at the next scheduled time at the recommended dose. The individual daily doses of nintedanib should not be increased beyond the recommended dose to makeup for missed doses. The recommended maximum daily dose of 400 mg should not be exceeded. Patients may continue therapy with nintedanib after discontinuation of docetaxel for as long as clinical benefit is observed or until unacceptable toxicity occurs.

For posology, methods of administration, and dose modifications of docetaxel, please refer to the corresponding product information for docetaxel. Dose adjustments as initial measure for the management of adverse reactions treatment with nintedanib should be temporarily interrupted until the specific adverse reaction has resolved to levels that allow continuation of therapy (to grade 1 or baseline). Nintedanib treatment may be resumed at a reduced dose. Dose adjustments in 100 mg steps per day (i.e. a 50 mg reduction per dosing) based on individual safety and tolerability are recommended as described in Table below. In case of further persistence of the adverse reaction(s), i.e. if a patient does not tolerate 100 mg twice daily, treatment with nintedanib should be permanently discontinued.

Recommended dose adjustments for nintedanib in case of diarrhoea, vomiting and other non-haematological or haematological adverse reactions:

| CTCAE* Adverse reaction | Dose adjustment |
|---|---|
| Diarrhoea ≥ grade 2 for more | After treatment interruption |
| than 7 consecutive days despite | and recovery to grade 1 or baseline. |
| anti-diarrhoeal treatment | dose reduction from 200 mg |
| OR | twice daily to 150 mg twice daily and - |
| Diarrhoea ≥ grade 3 despite | if a 2^{nd} dose reduction is considered |
| anti-diarrhoeal treatment | necessary - from 150 mg twice |
| Vomiting ≥ grade 2 | daily to 100 mg twice daily. |
| AND/OR | |
| Nausea ≥ grade 3 | |
| despite anti-emetic treatment | |
| Other non-haematological | |
| or haematological | |
| adverse reaction of ≥ grade 3 | |

*CTCAE: Common Terminology Criteria for Adverse Events

(see Web-site of the EMA for more detailed information:
http://www.ema.europa.eu/docs/en_GB/document_library/EPAR_-_Product_Information/human002569/WC500179970.df).

1. A method for a treating thyroid cancer or non-small cell lung cancer in a patient in need thereof, said method comprising the steps of
a) determining whether a RET fusion gene and/or a RET fusion gene product is present in a sample from said patient; and
b) selecting said patient for said treatment if said RET fusion gene and/or a RET fusion gene product is determined to be present in said sample, and
c) administering 3-Z-[1-(4-(N-((4-methyl-piperazin-1-yl)-methylcarbonyl)-N-methyl-amino)-anilino)-1-phenyl-methylene]-6-methoxycarbonyl-2-indolinone, or a pharmaceutically acceptable salt thereof, to said patient.

2. A method for predicting the clinical outcome of a thyroid cancer or non-small cell lung cancer treatment of a patient with the compound 3-Z-[1-(4-(N-((4-methyl-piperazin-1-yl)-methylcarbonyl)-N-methyl-amino)-anilino)-1-phenyl-methylene]-6-methoxycarbonyl-2-indolinone or a pharmaceutically acceptable salt thereof, said method comprising a step of determining whether a RET fusion gene and/or a RET fusion gene product is present in a sample from said patient.

3. The method of claim 1, wherein the compound is the monoethanesulphonate salt form of 3-Z-[1-(4-(N-((4-methyl-piperazin-1-yl)-methylcarbonyl)-N-methyl-amino)-anilino)-1-phenyl-methylene]-6-methoxycarbonyl-2-indolinone.

4. (canceled)

5. (canceled)

6. The method of claim 1, wherein the sample is a tumor biopsy of said cancer obtained from said patient.

7. The method of claim 1, wherein the sample is blood obtained from said patient or a blood fraction obtained from said patient.

8. The method of claim 7, wherein the sample is blood obtained from said patient.

9. The method of claim 1, wherein said RET fusion gene is CCDC6-RET, and wherein said RET fusion gene product is the product of the CCDC6-RET fusion gene.

10. The method of claim 1, wherein said cancer is non-small cell lung cancer.

11. The method of claim 1, wherein said cancer is thyroid cancer.

12. The method of claim 1, wherein said treatment is a monotherapy with said 3-Z-[1-(4-(N-((4-methyl-piperazin-1-yl)-methylcarbonyl)-N-methyl-amino)-anilino)-1-phenyl-methylene]-6-methoxycarbonyl-2-indolinone or said pharmaceutically acceptable salt thereof.

13. The method of claim 1, wherein said treatment is a combination therapy
comprising an additional anticancer therapy.

14. The method of claim 1, wherein said RET fusion gene and/or RET fusion gene product is the fusion gene CCDC6-RET.

15. The method of claim 1, wherein said step of determining comprises a step of
forming a detection complex of said RET fusion gene with a reagent for detecting said RET fusion gene, said reagent being a nucleic acid capable of specific binding to said RET fusion gene;
and/or wherein said step of determining comprises a step of
forming a detection complex of said RET fusion gene product with a reagent for detecting said RET fusion gene product, wherein said RET fusion gene product is a fusion protein, and wherein said reagent is an antibody capable of specific binding to said fusion protein;
and/or wherein said step of determining comprises a step of
forming a detection complex of said RET fusion gene product with a reagent for detecting said RET fusion gene product, wherein said RET fusion gene product is an mRNA of the RET fusion gene, and wherein said reagent is at least one nucleic acid capable of specific binding to said mRNA of the RET fusion gene.

16. The method of claim 1, wherein said method is an in vitro method.

17-34. (canceled)

35. The method of claim 13, wherein said additional anticancer therapy is with docetaxel.
